(19) United States
(12) Patent Application Publication
(10) Pub. No.: US 2006/0217425 A1
(43) Pub. Date: Sep. 28, 2006

(54) Methods of treating cancer using PPAR-gamma antagonists

(76) Inventors: Jack Burton, Long Island City, NY (US); Rosalyn Blumenthal, West Orange, NJ (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(21) Appl. No.: 11/376,598

(22) Filed: Mar. 14, 2006

Related U.S. Application Data
(60) Provisional application No. 60/661,024, filed on Mar. 14, 2005.

(51) Int. Cl. A61K 31/44; A61K 31/165

(52) U.S. Cl. 514/352; 514/619

(57) ABSTRACT

The present invention relates to compositions and methods of using peroxisome proliferator-activated receptor-gamma (PPARγ) antagonists. In one embodiment, the present invention relates to compositions and methods for preparing and using such antagonist compositions. In another embodiment, the present invention provides for using PPAR-gamma antagonist compositions to treat disease such as cancer. In other embodiments, the present invention provides for using PPAR-gamma antagonist and one or more additional anti-cancer agent compositions to treat cancer.

### CROSS REFERENCE TO RELATED APPLICATIONS

This application claims the benefit under 35 U.S.C. §119(e) of provisional U.S. patent application Ser. No. 60/661,024 filed on Mar. 14, 2005. The aforementioned application is hereby incorporated by reference in its entirety for all purposes.

### FIELD

The present invention relates to compositions and methods of using peroxisome proliferator-activated receptor-gamma (PPARγ) antagonists. In one embodiment, the present invention relates to compositions and methods for preparing and using such antagonists. In another embodiment, the present invention provides for using PPARγ antagonist compositions to treat disease such as cancer.

### BACKGROUND

Three PPARs are known: PPARα, PPARδ and PPARγ. These are encoded by different genes and 2 isoforms of PPARγ are known to exist: PPARγ1, and PPARγ2.

Biological processes known to be modulated by PPARγ include, for example, cell differentiation to produce lipid accumulating cells, regulation of insulin sensitivity and blood glucose levels, which are involved in hyperglycemia, hypoglycemia/hyperinsulinism (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation that leads to the formation of atherosclerotic plaques, inflammatory response, carcinogenesis, hyperplasia, and adipocyte differentiation.

Peroxisomes are cellular organelles which play a role in controlling the redox potential and oxidative stress of cells by metabolizing a variety of substrates such as hydrogen peroxide. There are a number of disorders associated with oxidative stress. For example, inflammatory response to tissue injury, pathogenesis of emphysema, ischemia-associated organ injury (shock), doxorubicin-induced cardiac injury, drug-induced hepatotoxicity, atherosclerosis, and hyperoxic lung injuries, are each associated with the production of reactive oxygen species and a change in the reductive capacity of the cell. It been suggested that that PPARγ activators (agonists) that control the redox potential and oxidative stress in cells may be effective in the treatment of such disorders.

In addition, PPARγ receptor subtypes are involved in activating adipocyte differentiation, but are not involved in stimulating peroxisome proliferation in the liver. In this scheme, activation of PPARγ has been implicated in adipocyte differentiation through stimulation of adipocyte-specific gene expression

In one study, PPARγ agonists such as troglitazone have been shown to convert cancerous tissue to normal tissue in liposarcoma, a tumor of fat. It also has been suggested that PPARγ activators maybe useful in the treatment of breast and colon cancer (Proc. Nat'l Acad. Sci USA (1998) 95:8806-8811, Nature Medicine (1998) 4:1046-1052).

### SUMMARY

One embodiment of the present invention provides for novel methods of treating a cancer in a subject by administering to the subject an effective amount of a PPARγ antagonist composition. In accordance with this method, the cancer may be an epithelial or non-epithelial cancer but not a sarcoma Some of these cancers include but are not limited to pancreatic cancer, ovarian cancer, prostate cancer, renal cancer, testicular cancer, urothelial cancer skin cancer, melanoma, colon cancer, kidney cancer, brain cancer or a hematopoietic cancer. Hematopoietic cancers include, for example, lymphoma, multiple myeloma and leukemia.

In some embodiments, a PPARγ antagonist may be a compound having the formula:

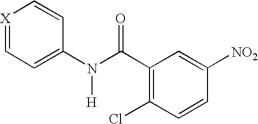

where X can be a CH or N. In one embodiment, X may be a CH In accordance with this embodiment, the PPARγ antagonist is GW9662. In another embodiment, X may be a N. In accordance with this embodiment, the PPARγ antagonist is T0070907.

In one embodiment, a T0070907 composition may be used to treat cancer that may include but are not limited to breast cancer, pancreatic cancer, ovarian cancer, prostate cancer, renal cancer, testicular cancer, urothelial cancer skin cancer, melanoma, colon cancer, kidney cancer, brain cancer or a hematopoietic cancer. Hematopoietic cancers include, for example, lymphoma, multiple myeloma and leukemia. In another embodiment, compositions of GW9662 and T0070907 may be used to treat any one of the cancers included above.

In other embodiments of the invention, a PPARγ antagonist composition may be used to treat a subject having cancer in combination with another agent such as another anti-cancer agent. In accordance with this embodiment the anti-cancer agent may include but is not limited to an antibody, an immunoconjugate, an antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, and an immunomodulator. In other embodiments, the anti-cancer agent may include but is not limited to cyclophosphamide, etoposide, vincristine, procarbazine, carmustine, doxorubicin, methotrexate, bleomycin, and dexamethasone. Inmunomodulators include but are not limited to interferons, lymphokines, cytokines, and growth factors. In one particular embodiment, a PPARγ antagonist composition may be used to treat a subject in combination with other nuclear hormone superfamily member. In accordance with this embodiment a nuclear hormone superfamily member may include but is not limited to a retinoid-X-receptor, estrogen receptor, progesterone receptor, androgen receptor, vitamin D receptor, retinoic acid receptor, pregnane-X-receptor, and thyroid hormone receptor.

### DETAILED DESCRIPTION

In the following section, several methods are described to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description in order to prevent unnecessary masking of the various embodiments.

PPARγ is a ligand-regulated transcription factor of the nuclear hormone receptor superfamily. It is expressed in certain normal tissues such as adipose tissue. PPARγ is expressed in a variety of cancers such as a wide range of epithelial and hematopoietic cancers, as well as melanoma and primary brain cancer. PPARγ agonists like troglitazone have been shown to inhibit the in vitro and in vivo growth of selected epithelial cancer cell lines.

Yet in other studies presented herein, PPARγ antagonists not agonists may be more effective in the treatment of certain cancers. Details herein reveal that PPARγ antagonists are surprisingly effective in treating a variety of cancers (See Example Section) when compared to the agonist.

One embodiment of the present invention provides for novel methods of treating a subject having or suspected of developing a cancer by administering to the subject an effective amount of a PPARγ antagonist composition. In accordance with this method, the cancer may be an epithelial or non-epithelial cancer but not a sarcoma. Some of these cancers include but are not limited to breast cancer, pancreatic cancer, ovarian cancer, prostate cancer, renal cancer, testicular cancer, urothelial cancer skin cancer, melanoma, colon cancer, kidney cancer, brain cancer or a hematopoietic cancer. Hematopoietic cancers include, for example, lymphoma, multiple myeloma and leukemia.

In one embodiment of the present invention, PPARγ antagonists may be used to treat cancer in a subject suffering from cancer. In a more particular embodiment, PPARγ antagonists T0070907 and/or GW9662, whose structures are shown below may be used to treat a subject suffering from cancer:

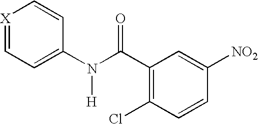

In one embodiment, when X is CH, the PPARγ antagonist is called GW9662. In another embodiment, when X is N, the PPARγ antagonist is called T0070907.

GW9662 and T0070907 are known in the art and are commercially available from, for example, Sigma-Aldrich (St. Louis, Mo.) and Cayman Chemical Co. (Ann Arbor, Mich.).

In another embodiment, a pharmaceutical composition may further contain one or more additional binding molecules to identify and/or treat a certain cell population (e.g. a tumor cell population) which specifically bind to one or more antigens selected from the group consisting of CD4, CD5, CD8, CD14, CD15, CD19, CD20, CD21, CD22, CD23, CD25, CD30, CD33, CD37, CD38, CD40, CD40L, CD46, CD52, CD54, CD66 (a,b,c,d), CD74, CD80, CD126, CD138, CD154, B7, MUC1, MUC2, MUC3, MUC4, MUC16, HLA-DR, HM1.24, tenascin, VEGF, EGFR, CEA, CSAp, ILGF, placental growth factor, Her2/neu, carbonic anhydrase IX, IL-6, SI00, MART-1, TRP-1, TRP-2, gp100, amyloid and combinations thereof, where the additional binding molecule is given before, with, or after any pharmaceutical composition disclosed herein containing a PPARγ antagonist.

Within any embodiment disclosed herein, it is contemplated that a PPARγ antagonist may include a compound that has the potential to inhibit the ability of PPARγ agonists to stimulate the receptor/transcription factor. Although, a number of compounds are known to bind to PPARγ and can have both partial agonist and antagonist activities, the embodiments of the present invention do not include these compounds.

Combination Therapies

In one embodiment, any PPARγ antagonist disclosed herein may be administered alone or in combination with other antagonists and/or other agents. When used in combination, the PPARγ antagonists may be administered together, sequentially, or in any order. Additional anti-cancer drugs may be used as described and the agents may be administered together or in any order.

In one embodiment, the anti-cancer agents used in combination with one or more PPARγ antagonist include but are not limited to antibody, an immunoconjugate, and antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, and an immunomodulator.

In other embodiments, the anti-cancer agents used in combination with one or more PPARγ antagonist include but are not limited to cyclophosphamide, etoposide, vincristine, procarbazine, carmustine, doxorubicin, methotrexate, bleomycin, and dexamethasone.

In other embodiments of the present invention, the anti-cancer agents used in combination with one or more PPARγ antagonist include but are not limited to interferons (e.g. IFN-yγ, β and/or α), lymphokines, cytokines (e.g. interleukin-2 (IL-2), IL-18, IL-11), and growth factors (e.g. platelet derived growth factor (PDGF), tumor necrosis factor (TNF) and epidermal growth factor (EGF)).

Agents or factors suitable for use in a combined therapy may be any chemical compound or treatment method that induces DNA damage when applied to a cell. Such agents and factors include radiation and waves that induce DNA damage such as γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use may include but are not limited to adriamycin, 5-fluorouracil (5FU), etoposide (VP-16), camptothecin, actinomycin-D, mitomycin C, cisplatin (CDDP) and even hydrogen peroxide. It is also contemplated herein that the use of a combination of one or more DNA damaging agents may be required depending on the subject and the condition of the subject, whether radiation-based or actual compounds, such as the use of X-rays with cisplatin or the use of cisplatin with etoposide.

In treating cancer according to the present invention, tumor cells may be contacted with an agent in addition to the antagonist. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition that may include a compound such as, adriamycin, 5-fluorouracil, etoposide, camptothecin, actinomycin-D, or mitomycin C. The agent may be prepared and used as a combined therapeutic composition, or kit, by combining it with one or more of the PPARγ antagonists, as described above.

Agents that directly cross-link nucleic acids, specifically DNA, are envisaged to facilitate DNA damage leading to a synergistic, antineoplastic combination. Agents such as cisplatin, and other DNA alkylating agents may be used.

Agents that damage DNA also include compounds that interfere with DNA replication, mitosis and chromosomal segregation. Such chemotherapeutic compounds include but are not limited to adriamycin, also known as doxorubicin, etoposide, cisplatin, carmustine, podophyllotoxin, and the like. Widely used in a clinical setting for the treatment of neoplasms, these compounds for example may be administered intravenously through bolus injections at doses ranging from 25-75 mg/m² at 21 day intervals for adriamycin, to 35-100 mg/m² for etoposide intravenously or double the intravenous dose orally.

Agents that disrupt the synthesis and fidelity of nucleic acid precursors and subunits also lead to DNA damage are contemplated herein. A number of nucleic acid precursors have been developed for this purpose. Particularly useful are agents that have undergone extensive testing and are readily available, such as 5-fluorouracil (5-FU). Although quite toxic, 5-FU is applicable in a wide range of carriers, including topical. However intravenous administration with doses ranging from 3 to 15 mg/kg/day is commonly used. Other agents include but are not limited to cytosine arabinoside, gemcitabine, and fludarabine.

Other factors that cause DNA damage and have been used extensively include γ-rays, X-rays, and/or the directed delivery of radioisotopes to tumor cells. Other forms of DNA damaging factors also are contemplated such as microwaves and UV-irradiation. It is most likely that all of these factors effect a broad range of damage to DNA, on the precursors of DNA, the replication and repair of DNA, and the assembly and maintenance of chromosomes. Dosage ranges for X-rays range from daily doses of 100-300 cGy or for prolonged periods of time (2-6 weeks), to single doses of 800-3000 cGy. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, and in particular to pages 624-652. Some variation in dosage may be necessary depending on the condition of the subject being treated. The healthcare professional will, in any event, determine the appropriate dose for the individual subject. Moreover, for human administration, preparations should meet sterility, pyrogenicity, and general safety and purity standards as required by the FDA Office of Biologics standards.

The regional delivery of antisense or expression constructs to patients with cancer will be a very efficient method for delivering a therapeutically effective gene to counteract the clinical disease. Similarly, chemo- or radiotherapy may be directed to a particular, affected region of the subject's body. Alternatively, systemic delivery of expression construct and/or the agent may be appropriate in certain circumstances, for example, where extensive metastasis has occurred.

It is contemplated that combination PPARγ antagonists and gene therapies may be advantageous. Any tumor-related gene conceivably can be targeted in combination with one or more PPARγ antagonists. For example, p21, p53, Rb, APC, DCC, BCL-2, NF-1, NF-2, p16, FHIT, WT-1, MEN-I, MEN-II, VHL, FCC, MCC, ras, myc, neu, raf, erb, src, fins, jun, trk, ret, gsp, hst, bcr and abl are examples of genes that may be targeted.

Administration

In one example, the PPARγ antagonist may be orally administered, for example, with an inert diluent or with an assimilable edible carrier, or it may be enclosed in hard or soft shell gelatin capsules, or it may be compressed into tablets, or it may be incorporated directly with the food of the diet. In one particular embodiment, a PPARγ antagonist composition may be administered orally to a subject having or suspected of developing a condition such as cancer. For oral therapeutic administration, the active compound may be incorporated with excipient and used in the form of ingestible tablets, buccal tablets, troches, capsules, elixirs, suspensions, syrups, wafers, and the like. Such compositions and preparations should contain at least 0.1% of the antagonist. The percentage of the compositions and preparations may be varied such that a suitable dosage will be obtained. Preferred compositions or preparations according to the present invention are prepared so that an oral dosage unit form contains between about 1 and 500 mg of active compound, although other dosage forms may be used. Suitable pharmaceutical compositions of the antagonists are known in the art.

In addition, it is contemplated herein that any PPARγ antagonist composition disclosed herein may be introduced to a subject in need of such a composition in a sustained release formula such as a formula designed to release once it reaches a target such as a target cell population (e.g. tumor cell population treated with a microparticle or nanoparticle formulation). In another embodiment, it is contemplated herein that any PPARγ antagonist composition disclosed in the present invention may be of a formula that releases all of the formula once it reaches a target cell population such as a tumor cell population. In one embodiment, the introduction of any PPARγ antagonist composition may precede, coincide or follow any other treatment such as a anti-cancer treatment.

In addition it is contemplated herein that any PPARγ antagonist composition disclosed herein may be used in a formula such as a gelatinous formula to coat the exterior or inner surface of another microparticle formulation or other anti-cancer agent formulation.

The antagonist may also be administered parenterally, intervenously, intraperitoneally, intramuscularly and subcutaneously. Solutions of an antagonist or a pharmacologically acceptable salt thereof (when appropriate) can be prepared in water suitably mixed with a surfactant such as hydroxypropyl-cellulose. Dispersion can also be prepared in glycerol, liquid polyethylene glycols, in oils and combinations thereof. Under ordinary conditions of storage and use, these preparations contain a preservative to prevent the growth of microorganisms.

A pharmaceutical form suitable for injectable use include sterile aqueous solutions or dispersions and sterile powders for the extemporaneous preparation of sterile injectable solutions or dispersions. In all cases, the form must be sterile and must be fluid to the extent that easy syringability exists. It may be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like) and suitable mixtures thereof. The proper fluidity can be maintained, for example, by the use of a coating such as lecithin, by the maintenance of the required particle size in the case of dispersion and by the use or surfactants. The prevention of the action of microorganisms can be brought about by various antibacterial and antifungal agents, for example, parabens, chlorobutanol, phenol, sorbic acid, thimerosal, and the like.

In other embodiments, sterile injectable solutions may be prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof

A health-care provider can determine the dosage of the therapeutic compositions described suitable for treatment of cancer and the dosage may vary with the form of administration and the particular antagonist chosen, and also will vary with the particular patient under treatment. The healthcare provider may begin treatment with small dosages by small increments until the optimum effect under the circumstances is reached. The therapeutic dosage will generally be from 0.1 to 100 mg/day or from about 0.1 mg to about 50 mg/kg of body weight per day, or 0.1 mg to about 30 mg/kg of body weight per day, or more preferably 10 mg to about 30 mg/kg of body weight per day, and higher, although it may be administered in several different dosage units. Higher dosages may be required for oral administration.

The antagonists and compositions containing the antagonists may be administered as frequently as necessary in order to obtain the desired anti-cancer effect. In accordance with this embodiment, the composition may be administered more than once a day, daily, every other day, 2 times per week, once a month, 2 times a month etc.

One skilled in the art will readily appreciate that the present invention is well adapted to carry out the objects of the invention and obtain the ends and advantages mentioned, as well as those inherent therein. The methods described above are merely exemplary and not intended as limitations on the scope of the present invention.

The embodiments are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### EXAMPLES

### Example 1

In one study, in vitro cytotoxicity of a PPARγ agonist with a favorable toxicity profile (pioglitazone [Actos®]) was tested against two PPARγ antagonists (T0070907 and GW9662) in a panel of solid tumor and hematopoietic cancer cell lines, using an MTT proliferation assay. Effects of the PPARγ agonist in combination with the antagonists or with chemotherapy drugs were also evaluated, and these results were correlated with PPARγ expression as assessed by RT-PCR and immunoblotting, using assay methods that are well known in the art.

Methods

In one exemplary method, stock solutions of the above referenced agents were dissolved in 50:50 DMSO:DMF and then diluted into cell growth medium. Concentrations of 2-40 micromolar over 5-7 days provided significant growth inhibition for a range of hematopoietic (NHL & MM) and epithelial lines (renal cell, colon, breast). Addition of IL-6 (Interleukin 6) to MM lines did not cause resistance to either antagonist as it does to some agents from other categories. Also, both antagonists were dissolved in a cyclodextrin-propylene glycol vehicle and administered to mice at doses of 7.5-15 mg/kg intraperitoneally daily for 3 weeks with mild-moderate toxicity. Surprisingly, combinations of GW9662 with the agonist (pioglitazone) and T0070907 with this agonist led to additive increases in growth inhibition of cancer cell lines rather than the expected antagonism.

The IC₅₀ for pioglitazone in solid tumor lines ranged from 11.1 μM to >120 μM (mean±SD=60.1±28.1). Hematopoietic (NHL and MM) cell lines appear to be less sensitive to this drug (IC₅₀=82.0−124.7; mean±SD=101.4±17.7). Both of the PPARγ antagonists were found to be growth inhibitory for both solid and hematopoietic lines. In addition, both PPARγ antagonists were more potent than pioglitazone with IC₅₀ values of 7.8 to 28.7 μM (T0070907 was more potent than GW9662).

### Example 2

In one exemplary method, three PPARγ ligands were combined in pilot studies, in which pioglitazone and either T0070907 or GW9662 showed additive effects.

### Example 3

In one exemplary method, all epithelial cancer lines tested expressed PPARγ by RT-PCR and the majority expressed the protein. With respect to hematopoietic lines, only the myeloid lines, U937 and K562, were positive by RT-PCR, and only K562 expressed PPARγ protein.

### Example 4

In one exemplary method, a 58-year old male patient presenting with multiple myeloma is treated with a PPARγ antagonist oral composition at a dose of about 10 mg/kg once daily for 4 weeks. After the treatment, the composition ameliorates the cancer in the patient.

In these exemplary methods, the data demonstrate novel, potent, and growth inhibitory effects of PPARγ antagonist drugs for treated neoplastic cells, such as epithelial and hematopoietic cells. This data demonstrated a greater overall potency than a PPARγ agonist. Both the PPARγ agonist and the antagonists are growth inhibitory for cancer cells independent of PPARγ expression levels. Moreover, the additive effects of combinations of agonist plus antagonist suggest non-overlapping mechanisms of action. These results confirm the effect of agonist drugs and reveal the potent inhibitory effect of PPARγ antagonists in cancer.

All of the COMPOSITIONS and/or METHODS and/or APPARATUS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS and/or APPARATUS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method of treating a cancer in a subject, comprising administering to a subject suffering from said cancer an effective amount of a PPARγ antagonist, wherein said cancer is selected from the group consisting of pancreatic cancer, ovarian cancer, prostate cancer, renal cancer, testicular cancer, urothelial cancer, skin cancer, melanoma, colon cancer, kidney cancer, breast cancer, brain cancer and hematopoietic cancer.

2. The method according to claim 1, wherein said PPARγ antagonist is a compound having the formula: 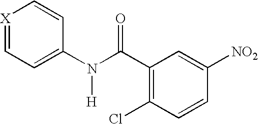
wherein X is CH or N.

3. The method according to claim 2, wherein said PPARγ antagonist is GW9662.

4. The method according to claim 2, wherein said PPARγ antagonist is T0070907.

5. The method according to claim 1, wherein said cancer is a cancer of the colon.

6. The method according to claim 1, wherein said cancer is a cancer of the kidney.

7. The method according to claim 1, wherein said cancer is lymphoma.

8. The method according to claim 1, wherein said cancer is multiple myeloma.

9. The method according to claim 1, wherein said cancer is a leukemia.

10. The method according to claim 1, further comprising administering to said subject a second anti-cancer agent.

11. A method of treating breast cancer in a subject comprising administering to said subject an effective amount of T0070907.

12. The method according to claim 11, further comprising administering to said subject a second anti-cancer agent.

13. The method according to claim 12, wherein said anti-cancer agent is selected from the group consisting of an antibody, an immunoconjugate, an antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, and an immunomodulator.

14. The method according to claim 12, wherein said anti-cancer agent is selected from the group consisting of cyclophosphamide, etoposide, vincristine, procarbazine, carmustine, doxorubicin, methotrexate, bleomycin, and dexamethasone.

15. The method according to claim 12, wherein said anti-cancer agent is an immunomodulator selected from the group consisting of interferons, lymphokines, cytokines, and growth factors.

16. The method according to claim 12, further comprising administering to said subject a third anti-cancer agent.

17. The method according to claim 16, wherein said third anti-cancer agent is selected from the group consisting of an antibody, an immunoconjugate, an antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, and an immunomodulator.

18. The method according to claim 16, wherein said anti-cancer agent is selected from the group consisting of cyclophosphamide, etoposide, vincristine, procarbazine, carmustine, doxorubicin, methotrexate, bleomycin, and dexamethasone.

19. The method according to claim 16, wherein said anti-cancer agent is an immunomodulator selected from the group consisting of interferons, lymphokines, cytokines, and growth factors.

20. A method of treating a cancer in a subject, comprising administering to a subject suffering from said cancer an effective amount of a combination of GW9662 and T0070907.

21. The method according to claim 20, wherein said cancer is selected from the group consisting of breast cancer, pancreatic cancer, ovarian cancer, prostate cancer, renal cancer, testicular cancer, urothelial cancer skin cancer, melanoma, colon cancer, kidney cancer, brain cancer and hematopoietic cancer.

22. A composition for treating a cancer in a subject, comprising a PPARγ antagonist and an anti-cancer agent, wherein said cancer is selected from the group consisting of pancreatic cancer, ovarian cancer, prostate cancer, renal cancer, testicular cancer, urothelial cancer skin cancer, melanoma, colon cancer, kidney cancer, breast cancer, brain cancer and hematopoietic cancer.

23. The composition according to claim 22, wherein said PPARγ antagonist is a compound having the formula: 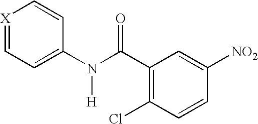
wherein X is CH or N.

24. The composition according to claim 23 wherein said PPARγ antagonist is GW9662.

25. The composition according to claim 23 wherein said PPARγ antagonist is T0070907.
